(19) United States
(12) Patent Application Publication
(10) Pub. No.: US 2009/0240318 A1
(43) Pub. Date: Sep. 24, 2009

(54) STENT EXPANSION COLUMN, STRUT AND CONNECTOR SLIT DESIGN

(75) Inventors: Aaron Chalekian, Minneapolis, MN (US); Joe Groff, Montrose, MN (US); Michael P. Meyer, Richfield, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/051,578

(22) Filed: Mar. 19, 2008

(51) Int. Cl. A61F 2/82; A61F 2/06

(52) U.S. Cl. 623/1.16; 623/1.35

(57) ABSTRACT

A bifurcated stent that is positionable adjacent to a branched body vessel. The stent includes a number of stent members at least some of which have slits extending through them. The slits are positioned relative to the tensile and compressive forces imposed by stent expansion to enhance the flexibility of the stent members. The slits can be positioned to increase stent compression about a catheter, increase how far the stent can be bent during catheter deployment, and allow specific portions of the stent to have improved flexibility. The slits can allow the stent to assume a bent shape, and compensate for rigidity imposed the bifurcating side branch. The slits can be combined with curved sections to create a highly flexibly stent.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use and more particularly to intravascular stents that include a plurality of cavities formed on one or more surfaces of the stent and are coated with drugs

2. Description of the Related Art

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable). Stents may be implanted to prevent restenosis following angioplasty in the vascular system.

A complication arises when stenoses form at vessel bifurcation sites. A bifurcation site is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All U.S. patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

At least one embodiment is directed to a bifurcated stent having a linear unexpanded state and a non-linear expanded state which comprises a generally tubular first body and a second body. The generally tubular first body defines a first lumen which is constructed out of a plurality of serially positioned strut columns interconnected by connector members. The strut columns are made up of strut members connected to adjacent strut members by peak members at their proximal end and by trough members at their distal ends. The first body has a side opening on a portion of the dorsal side of the first body, a pair of outer regions, and a medial region, the outer regions consist of a distal region distal to the side opening, and a proximal region proximal to the side opening. The second body comprises at least one projecting member which in the unexpanded state is positioned at least partially over the side opening and in the expanded state bends to define a second lumen in fluid communication with the first lumen.

At least one connector spanning between a medial region column and an outer region column has a slit. The slit is characterized as an absence of material extending along a length of the connector and opening onto at least one surface of the connector. The slit is at least partially defined by two sidewalls, each of the sidewalls extending from an outer surface at least partially down into the connector. In the unexpanded state the at least one slitted connector has a first shape and in the expanded state has a second shape, the first shape being more linear than the second shape. In the unexpanded state the two sidewall outer surfaces are substantially co-planar and in the expanded state they are non-co-planar.

At least one embodiment is directed to a stent in which the slitted connectors have a geometric displacement relative to each other in the unexpanded state which becomes deformed in the expanded state. The deformations being a compressing together of some sidewalls and a staggering of the outer surfaces of some of the sidewalls. The deformation can also being a twisting of the sidewalls around each other. Every connector in one region can be so deformed. Struts can also be slitted including struts located between a peak and a trough neither of which is are engaged to a connector.

At least one embodiment is directed to a stent in which the strut members of a high ratio portion of the first body has a higher ratio of slitted strut members to non-slitted strut members than all other portions of the first body. The high ratio portion (including but not limited to the medial region) can become the pinched portion of the bend in the stent and that portion of the stent on the opposite side of a circumferential cross section of the that portion of the stent having the highest density of slitted struts forms the tensed portion of a bend in the stent. The slit can extend past through a peak and be in both a strut and a connector. The slit has one configuration selected from the list consisting of: rectangular shaped, rectangular shaped with diamond ends, serial rectangles, wavy, triangular, trapezoidal, hourglass shaped, dumbbell shaped, and serial dumbbell shaped.

At least one embodiment is directed to a stent in which there are no slits in any portion of the second body. At least one embodiment is directed to a stent in which the slit is positioned to be exactly centered along the neutral axis. At least one embodiment is directed to a stent in which the central axis is offset from the neutral axis by a proportion relative to the distance between the closest side of the member and the neutral axis.

At least one embodiment is directed to a stent in which the second body comprises a plurality of generally linear spoke struts engaged at a first end to the ostial frame and whose second end is positioned away from the ostial frame. Spanning between the spokes are rings, in the unexpanded state the rings are in an undulated configuration and are positioned in concentric circles of differing distance from the ostial frame. In the expanded state, the rings are at least partially straightened out, the spoke struts bend up away from the side opening, and the spoke struts and rings define a wall of a second lumen in fluid communication with the first lumen. At least one spoke strut has a slit. In the unexpanded state the at least one slitted spoke strut has a first shape and in the expanded state has a second shape, the first shape being more linear than the second shape. A slit can also be positioned on the ostial ring non-inclusively between the engagement points with two spoke struts also has a slit extending through it.

At least one embodiment is directed to a stent in which the spoke strut has a turning portion positioned along the spoke strut length between the ring closest to the ostial frame and a next serially adjacent ring. The turning portion comprises two generally straight portions orthogonal to and engaged to the generally linear portion of the spoke strut and a curved portion linking the generally straight orthogonal portions. Each of the generally straight orthogonal portions have a slit extending through them and another slit extends from a position on the spoke closer to the first end to one of the generally straight orthogonal portions. Another slit extends from a position closer to the second end of the spoke strut to the other generally straight orthogonal portion.

This and other aspects of the invention are described in more detail in the accompanying description and drawings.

### BRIEF DESCRIPTION OF THE SEVERAL VIEWS OF THE DRAWINGS

The invention is best understood from the following detailed description when read in connection with accompanying drawings, in which:

FIG. 1 is a lateral cross sectional image of an unexpanded bifurcated stent in having slits in stent members.

FIG. 2 is a perspective image of an unexpanded bifurcated stent in having slits in stent members.

FIG. 3 is a lateral cross sectional image of an expanded bifurcated stent in having slits in stent members.

FIG. 4 is a perspective close up image of an expanded bifurcated stent in having slits in stent members.

FIG. 5 is a PRIOR ART stent member affected by tensile and compressive force moments.

FIG. 6 is a stent member undergoing tensile and compressive force moments with a slit relieving the stress moments.

FIG. 6A is a perspective view of a slitted stent member.

FIG. 7 is a stent member having a proportionally positioned slit.

FIG. 8 is a perspective view of slitted stent in an expanded state.

FIG. 9 is an image of slitted stent member in a staggered configuration.

FIG. 9A is a perspective view of a slitted stent member in a staggered configuration.

FIG. 10 is an image of a slitted stent member in a diverging configuration.

FIG. 11A is an image of a slitted stent member in a twisted configuration.

FIG. 11 is an image of a slitted stent member in a converging configuration.

FIG. 12 is stress diagram of an unslitted connected PRIOR ART stent columns.

FIG. 13 is stress diagram of connected stent columns with a slit in the connector.

FIG. 14A is stress diagram of connected stent columns with a slit in both stent columns.

FIG. 14B is stress diagram of connected stent columns with a slits in both the stent columns and the connector.

FIG. 15A is a graph of the stress levels of FIGS. 12, 13, 14A, and 14B.

FIG. 15B is a data table of the stress stress levels of FIGS. 12, 13, 14A, and 14B.

FIG. 16 is an overhead diagram of a slitted stent having a leftward yawing bend at its proximal side.

FIG. 17 is an overhead diagram of a slitted stent having leftward yawing bends at its proximal and distal sides.

FIG. 18 is a lateral view of a slitted stent having a dorsally directed pitching bend with the side branch assembly at the apex of the bend.

FIG. 19 is a flat pan view of an unexpanded bifurcated stent with slits in the medial region, connectors, and non-connected struts.

FIG. 19A is head on cross sectional view of a stent with slits in four quadrants of a stent portion.

FIG. 20 is a close up view of an unexpanded bifurcated stent with slits in the medial region, connectors, and non-connected struts.

FIG. 21 is a flat pan view of an unexpanded bifurcated stent with slits in the connectors.

FIG. 22 is a close up view of an unexpanded bifurcated stent with slits in the connectors.

FIG. 23 is a flat pan view of an unexpanded bifurcated stent with slits in the medial region, connectors, non-connected struts and the ostial frame.

FIG. 24 is a close up view of an unexpanded bifurcated stent with slits in the medial region, connectors, non-connected struts and the ostial frame.

FIG. 25 is a flat pan view of an unexpanded side branch assembly with slits in the spokes.

FIG. 26 is a flat pan view of an unexpanded side branch assembly with slits in the spokes and the rings.

FIG. 27 is a perspective view of interconnected columns with slits along the inner surface, outer surface and side faces of the members.

FIG. 28 is a flat pan view of stent struts with rectangular slits.

FIG. 29 is a perspective view of stent struts with diamond shaped slit ends.

FIG. 30 is a flat pan view of stent struts with diagonal rectangular slits.

FIG. 31 is a flat pan view of stent struts with off-center rectangular slits.

FIG. 32 is a flat pan view of stent struts with dual parallel slits.

FIG. 33 is a flat pan view of stent struts with multiple parallel offset slits.

FIG. 34 is a flat pan view of stent struts with wave shaped slits.

FIG. 35 is a flat pan view of stent struts with triangular slits.

FIG. 36 is a flat pan view of stent struts with trapezoidal slits.

FIG. 37 is a flat pan view of stent struts with hourglass slits.

FIG. 38 is a flat pan view of stent struts with dumbbell slits.

FIG. 39 is a flat pan view of stent struts with serial dumbbell slits.

FIG. 40 is a flat pan view of stent struts with peak/trough slits.

FIG. 41 is a flat pan view of stent struts with offset parallel slits in the connectors.

FIG. 42 is a flat pan view of stent struts with serial slits extending from the connector into the struts.

FIG. 43 is cross sectional view of a radially widening stent member with a radially straight slit.

FIG. 44 is cross sectional view of a radially widening stent member with a radially widening slit.

FIGS. 45-47 are flat pan views of unexpanded bifurcated stents with connectors in various portions of the medial region.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

For the purposes of this disclosure, like reference numerals in the figures shall refer to like features unless otherwise indicated.

Depicted in the figures are various aspects of the invention. Elements depicted in one figure may be combined with, or substituted for, elements depicted in another figure as desired.

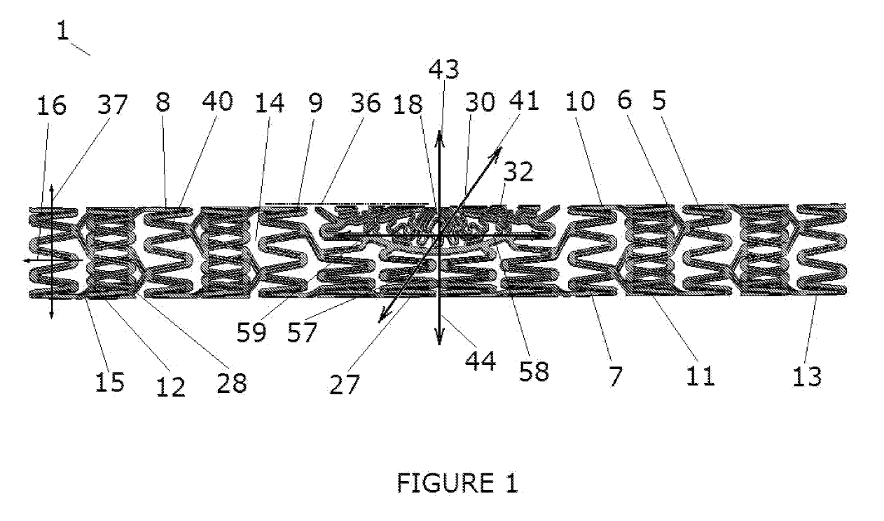

Referring now to FIG. 1, there is shown a bifurcated stent (1) in a linear unexpanded state. The stent comprises a number of stent members (8) including but not limited to: struts (7), strut connectors (6), and petals (32). One or more of the stent members (8) have one or more slits (40) within them. The slits (40) facilitate both stent introduction and stent implantation.

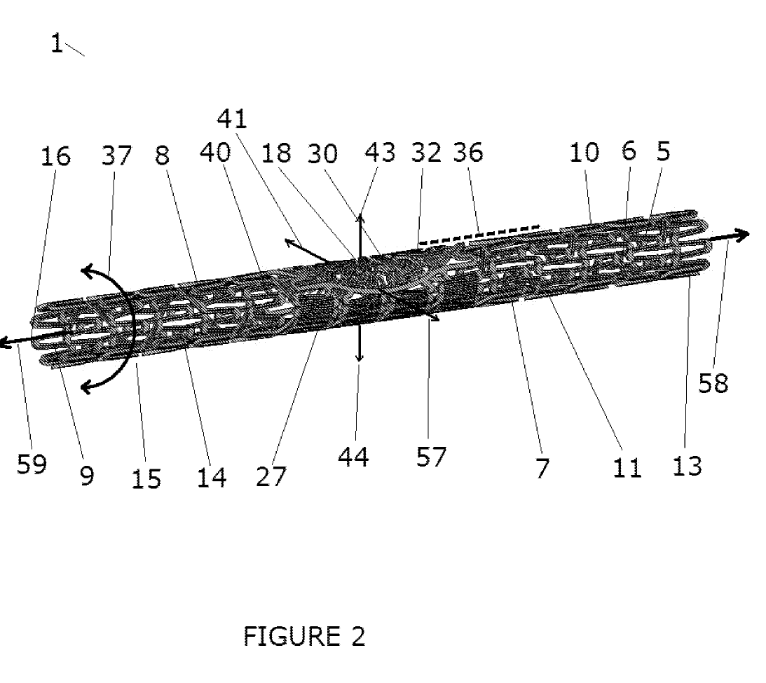

The stent members (8) combine to form two stent portions, a first body which is a generally tubular main stent body (10) and a second body which is a side branch assembly (30). Some examples of bifurcated stents contemplated by this invention include but are not limited to those found in commonly owned co-pending patent application Ser. No. 11/752837 which is incorporated by reference in its entirety. The slits (40) are located along one, some, or all of these two stent portions (10, 30). When in the unexpanded state (as can be seen for example in FIG. 2), the stent (1) is generally tubular in shape and its outer surface (4) defines a first circumferential layer (36).

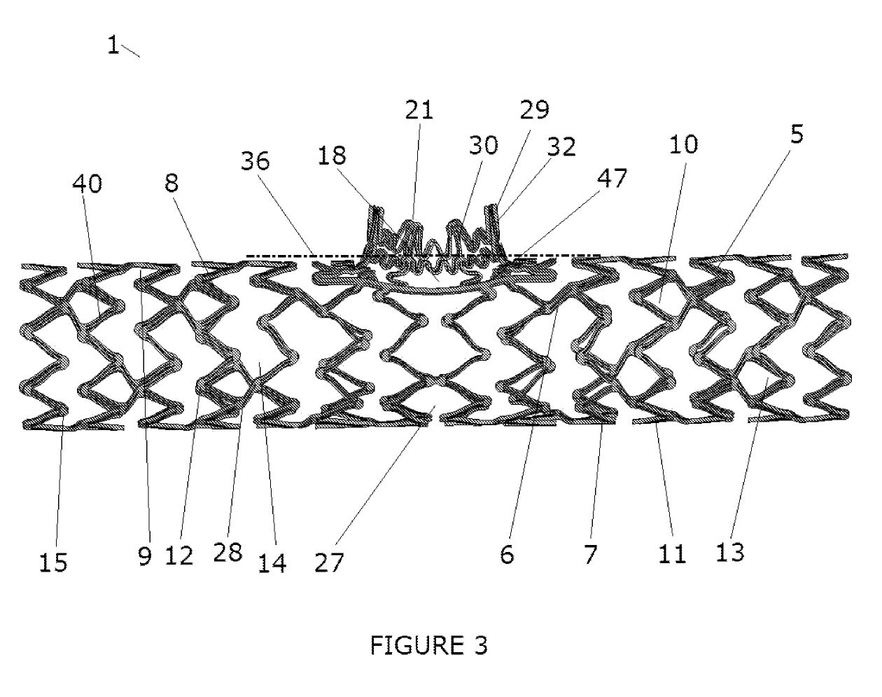

As shown in FIG. 3, when the side branch assembly (30) is deployed it forms a stent side branch (29) for stenting a body vessel that branches away from the main body vessel that the main stent body (10) stents. The side branch (29) extends from an ostial frame (47) radially to an oculus (21) or opening. Both the main stent body (10) and the side branch assembly (30) assume an expanded state using suitable techniques including self expansion, single or multiple balloon inflations, or by any other method known in the art. When in the expanded state, the stent (1) assumes a greater volume than when in the unexpanded state.

The main stent body (10) can comprise a number of stent members or struts (5). In some embodiments, the struts are joined by peaks (12) and troughs (28) to form columns (7) which in a circumferential direction (37) to form annular elements (11). The columns (7) are at least in part interconnected by connectors (6) and extend serially (i.e. one after the other along the longitudinal axis of the stent) form at least a part of the main stent body (10). The inner surface (9) of the main stent body (10) faces and defines a first fluid lumen (14). When the stent (1) is in the unexpanded state, at least a portion of the side branch assembly (30) generally lies along or within the first circumferential layer (36) and covers at least a portion of a side opening (18) present in the dorsal side of the main stent body (10). For purposes of this application the definition of term “dorsal” (43 in FIGS. 1 and 2) is in the direction vector which is radially directed away from the center of a cross section of the medial region of the stent towards the side opening of the stent, the definition of “ventral” (44 in FIGS. 1 and 2) is the diametrically opposite vector of dorsal, the definition of the term “dextral” (41 in FIGS. 1 and 2) is to the right of the dorsal-ventral axis when viewed from a proximal position, and the definition of the term “sinistral” (57 in FIGS. 1 and 2) is to the left of the dorsal-ventral axis when viewed from a proximal position. In the expanded state, at least a portion of the side branch assembly (30) such as for example projecting members or petals (32) bend, twist, extend and/or project dorsally away from the first circumferential layer (36) to form a side branch (29) which defines a secondary fluid lumen (34) which is in fluid communication with the first fluid (14).

The bifurcated stent (1) comprises a number of regions including an ostial region (17), a medial region (27), and two outer regions. The outer regions consist of a distal region (13) and a proximal region (15). For the purposes of this application, the definition of the term “ostial” is that portion of the stent which is located at the junction between the side branch assembly (30) and the main stent body (10). The ostial region (17) comprises the side branch assembly (30). The distal region (13) comprises that portion of the stent (1) distal to the ostial region (17). The proximal region (15) comprises that portion of the stent (1) proximal to the ostial region (17). The medial region comprises that portion of the stent not within the ostial region that is positioned between the distal (13) and proximal regions (15).

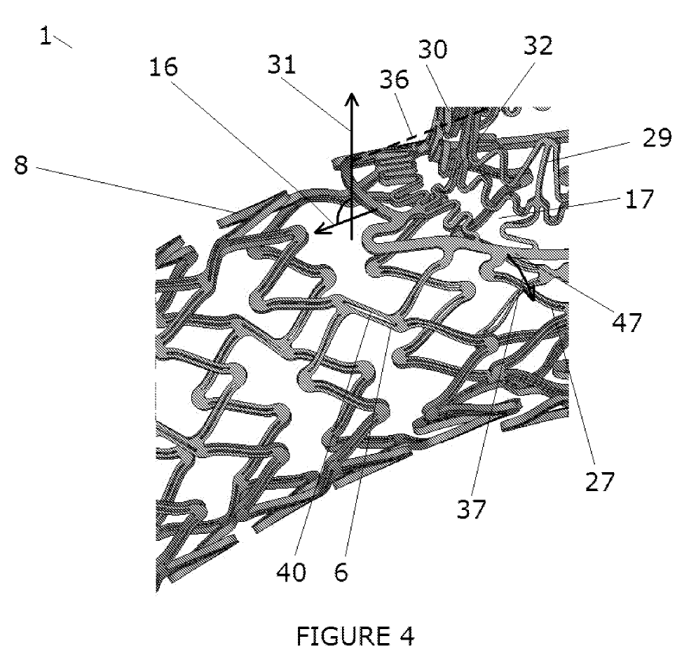

Referring now to FIG. 4 it can be seen that in at least one embodiment when the stent (1) is in the expanded state it becomes non-linear. In at least one embodiment expanded, each of the various regions undergo a significant amount of bending and flexing while transitioning from their unexpanded configuration to their expanded configuration. In particular the portions of the stent close to or within the ostial region (17) (such as the ostial frame (47) or petals (32)) undergo significant flexing as the side branch assembly (30) bends to rise up out of the circumferential layer (36). Some detailed examples of expansion stress and curved stent member designs which compensate for these expansion stresses are described in the commonly owned co-pending patent application Ser. No. 11/765679 the contents of which are incorporated by reference in their entirety. FIG. 4 illustrates an embodiment in which at least one connector (6) spanning between a medial region column and an outer region column has a slit (40) extending along its length.

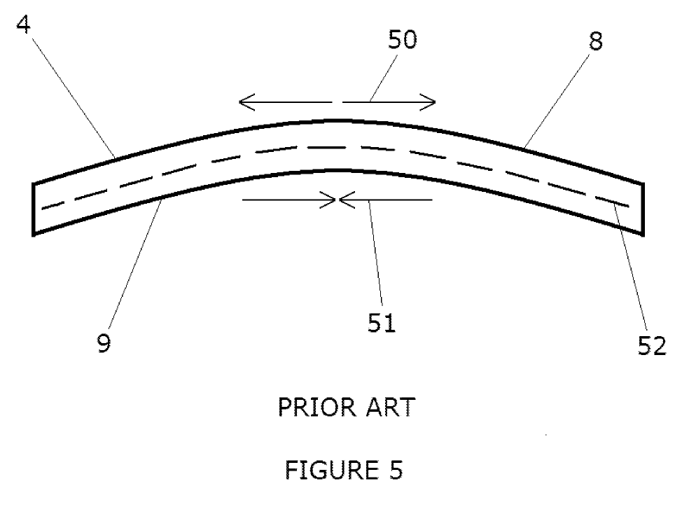

As the various stent members (8) bend into their expanded state configuration, different points on stent members (8) undergo different forms of stress. Referring to PRIOR ART FIG. 5 there is shown a bending stent member (8) which is extends in a purely circumferential direction (such as for example a circumferentially extending connector). When the member (8) is bent, the stent undergoes bending moments comprising a tensile stress moment and a compressive stress moment. The portion of the member closer to the outer surface (4) experiences an outward directed (positive) tensile stress moment (50) and the portion of the member (8) closer to the inner surface (9) in contrast experiences an inward directed (negative) compressive (51) stress moment. A neutral axis (52) which forms where the tensile (50) and compressive (51) moments cancel each other extends through a portion of the member (8) located between the outer (4) and inner surfaces (9) of the member (8). The location of the neutral axis (52) is not necessarily at the center of the member (8) nor is it necessarily linear and its orientation is dependent on both the material and shape of the member (8). The tensile stress moment (50) applies a force tending to pull the material(s) of the member (8) apart and the compressive stress moment (51) tends to push the material(s) of the member (8) together. In order to properly bend, both bending moments must be able to occur.

If the member (8) however is not sufficiently elastic, the oppositely directed bending moments (50, 51) can destructively interfere with each other. Interference between the bending moments can result in reduced flexibility in the members (8) limiting how far in a radial direction (31) the member (8) can be expanded. Interference between the moments (50, 51) can also result in shearing failures which can cause fractures in the member (8) in response to strong bending forces. Risk of shearing failures reduces the amount of radial expansion the stent can be safely expected to undergo. For purposes of this application the definition of the term “radial” means distance relative to the center of a circumferential cross section of the stent. Radial includes but is not limited to in the direction of the dorsal (43), ventral (44), sinistral (57), or dextral (41) vectors and/or according to a non-perfectly proximal or distal vector.

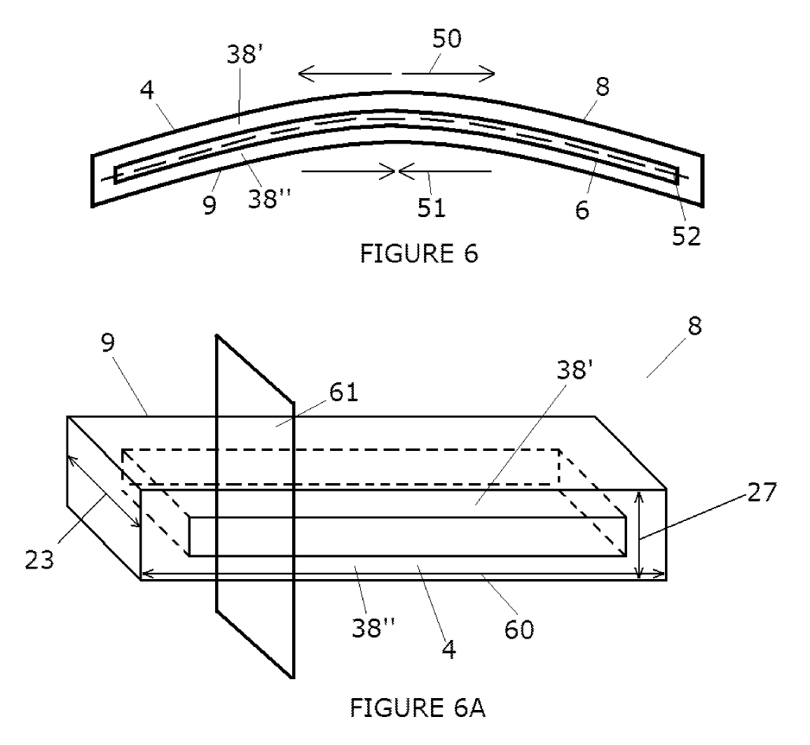

Referring now to FIG. 6 it is shown that the overall elasticity of the stent member (8) can be increased by separating at least some of the portion of the member (8) undergoing tensile stress and that portion of the member undergoing compressive stress with a void or slit (6) in between the two portions. The slit (6) is an absence of solid stent member material which separates member material undergoing tensile and compressive pressure. An embodiment is shown in FIG. 6A where the slit (40) is defined by a first sidewall (38′) on one side of the void and a second sidewall (38″) on the opposite side of the void. The slit extends along the length (60) of the member (8) and opens outward onto the outer surface (4) (the side not-facing the first lumen) of the member (8). The slit extends down from the outer surface (4) into the thickness (23) of the member (8) in the direction of the first lumen. The slit (40) may or may not have a second opening at the inner surface (9) of the member (8). The definition of thickness is measure of the distance between the outer and inner surfaces of the stent member. The width (27) of the member (the distance along the circumferential layer) determines how large the slit can be. The separation the slit (40) provides allows the material to move further in opposite respective directions than it would otherwise be able to move if the opposite sides of the slit (6) were in direct physical connection. In at least one embodiment (as shown in FIG. 6) the central vector of the slit extends exactly along the neutral axis (52). In at least one embodiment, the outer surfaces of the sidewalls (38a′, 38a″) are co-planar meaning that they are both commonly intersected by an intersecting plane (61).

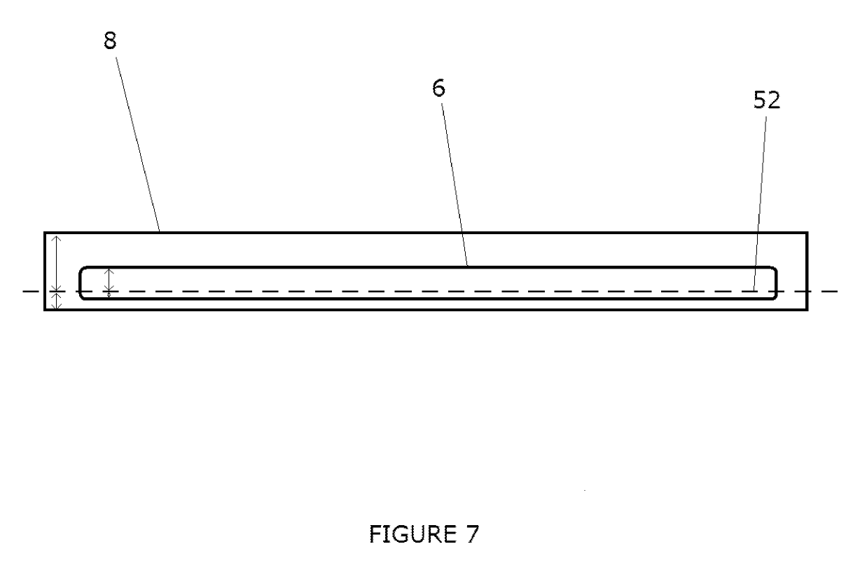

In at least one embodiment the dimensions of the slit (6) are proportional to the distance between that side of the neutral axis and the surface. As can be seen in FIG. 7 for example if the neutral axis (52) is located at a ¼:¾ position i.e. in a position where ¼ of the member's width or thickness is on one side of the neutral axis (52) and where ¾ of the member's width or thickness is on the other side of the neutral axis (52), the dimensions of the slit would be also be in a ¼:¾ position i.e. they define an aperture in which ¼ of the slit's width is on one side of the axis and in which ¾ of the slit's width is on the other side of the neutral axis (52). The proportions need not be exactly relational so a stent member having a ¼:¾ position relative to the neutral axis (52) could be combined with a slit positioned at a ⅓: 3/3 location relative to the neutral axis (52) or vice versa. In at least one embodiment the member (8) is constructed out of a material more likely to fail under compressive stress than under tensile stress and the slit proportions are designed to have more material on the tensile side than the compressive side. Some examples of materials more resistant to tensile stress are stainless steel, alloys of stainless steel, ductile materials, and any combination thereof In at least one embodiment the member is constructed out of a material more likely to fail under tensile stress than under compressive stress and the slit proportions are designed to have more material on the compressive side than the tensile side. Some examples of materials more resistant to compressive stress are cobalt chromium, nitinol, polymeric material, brittle materials and any combination thereof Referring again to FIG. 4 it can be seen that when the stent (1) is expanded, many of the stent members (8) are bent along more than one dimension. For example, as the ostial frame (47) is moved into the expanded configuration, the frame bends in a radial direction (31) as well as in longitudinal (16) and circumferential (37) directions. In addition the portions of the ostial frame (47) facing the medial region (27), bend in a direction opposite that which the petals (32) bend when they form the side branch (29). In addition because most members (8) do not extend in only a circumferential direction, their expansion has radial and circumferential components to them. These multi dimensional changes impose a number of directional forces on the struts which impose a multitude of contradictory compressive and expansive moments on different parts of the members (8) and significantly alter the vector of the neutral axis often causing the neutral axis to adopt a highly non-linear configuration. The strain imposed on the expanding stent members by these multi directional forces can be relieved by a number of embodiments having various kinds of slits (40) in various stent members (8).

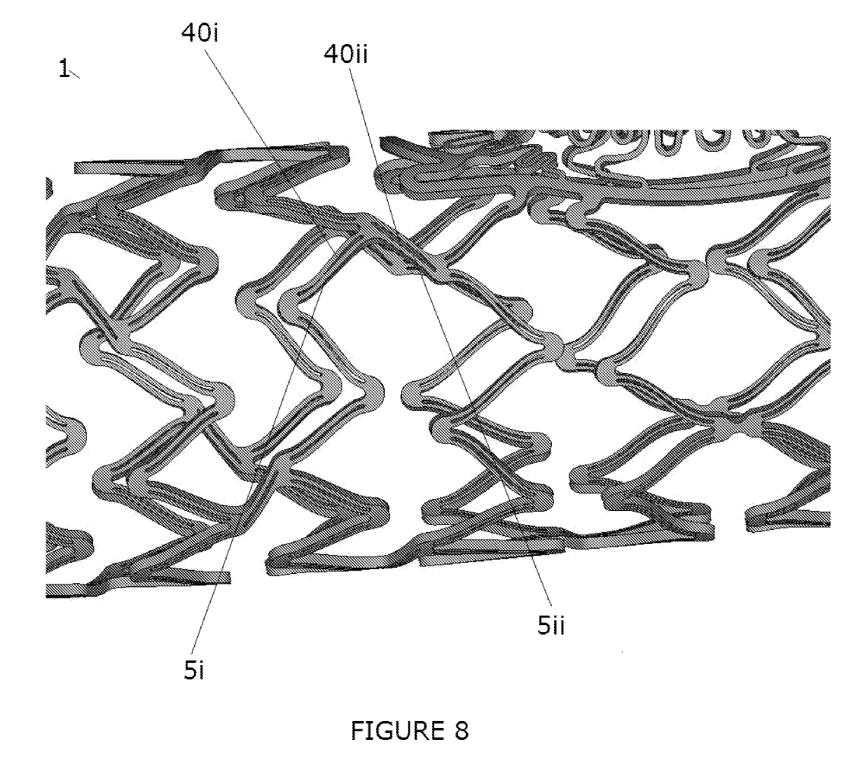

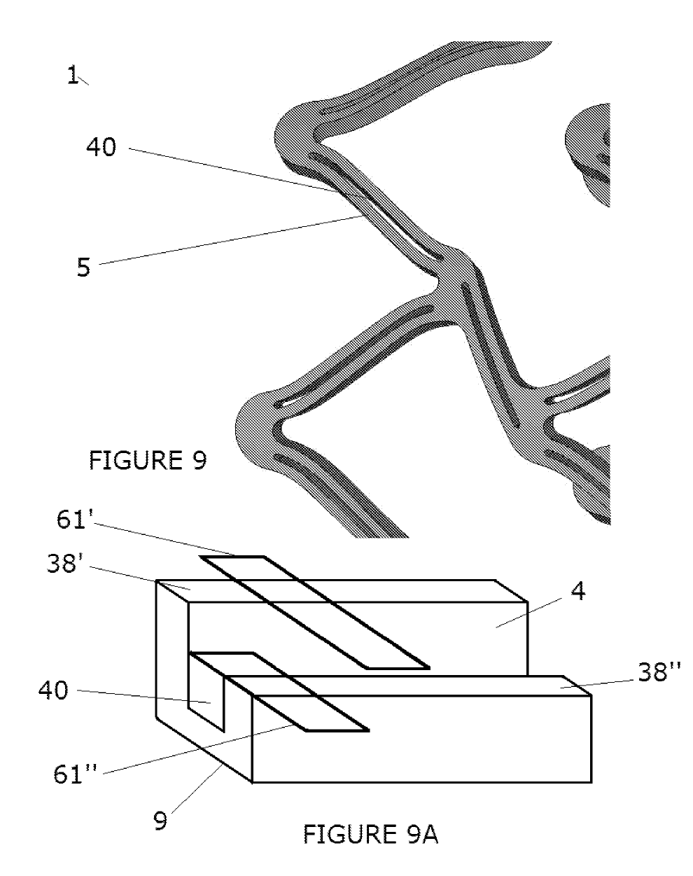

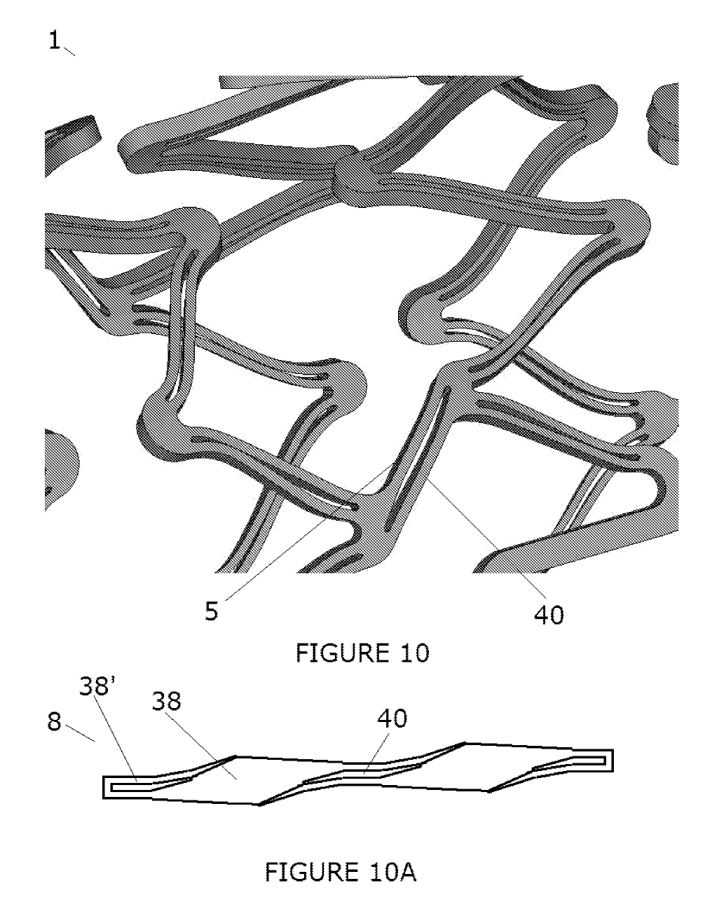

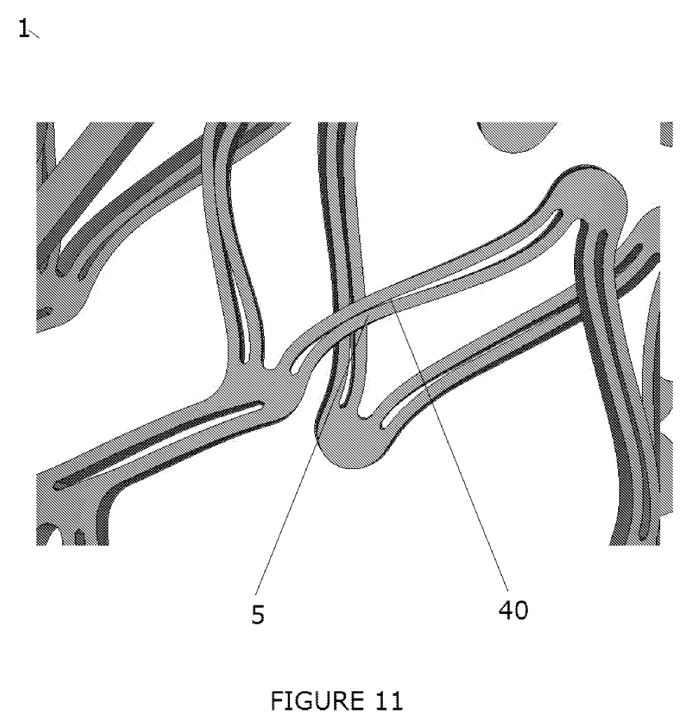

Referring now to FIG. 8 there shown is at least one embodiment in which the slits facilitate the expansion of the stent by allowing portions of the stent members to change from a more linear first shape to a less linear second shape. Some examples of the less linear second shapes include but are not limited to staggered, converged, diverged, and/or twisted relative to each other. In some positions in the stent (for example stent strut (5i)) the expansion stresses are somewhat relieved by allowing member portions to converge together. In other positions in the stent (for example stent strut (5ii)) the expansion stresses are somewhat relieved by allowing member portions to become staggered or out of alignment with each other. For purposes of this application, the definition of “staggered” is two objects each of which have a surface which at one point was coplanar to the other's surface but which are subsequently non-coplanar. The various converging, diverging, and staggering motions allow the member to respond to multidimensional force vectors in ways that stent members lacking the slits cannot. FIGS. 9 and 9A illustrates a slit allowing the member portions to stagger. FIG. 9A is a close up of a portion of the member shown in FIG. 6A. In it the sidewall surfaces (38a′ 38a″) are no longer intersected by a common plane and only intersect non-common planes (61′, 61″) so are non-co-planar. FIG. 10 shows the slit (40) allowing the member portions to diverge and FIG. 11 shows the slit allowing the member portions to converge.

Referring now to FIG. 10A there is shown at least one embodiment in which the sidewalls (38) of a stent member (8) are twisted around each other when the stent is expanded. When twisted, surfaces that in the unexpanded state have one geometric position relative to the outer or inner surface of the stent, occupy multiple geometric positions. For example, in FIG. 10A, when the sidewalls are twisted, the top of the sidewall (38′) at some portions along the length facing the inner surface of the stent, at some portions face the outer surface of the stent, and in some positions are in between these two extremes.

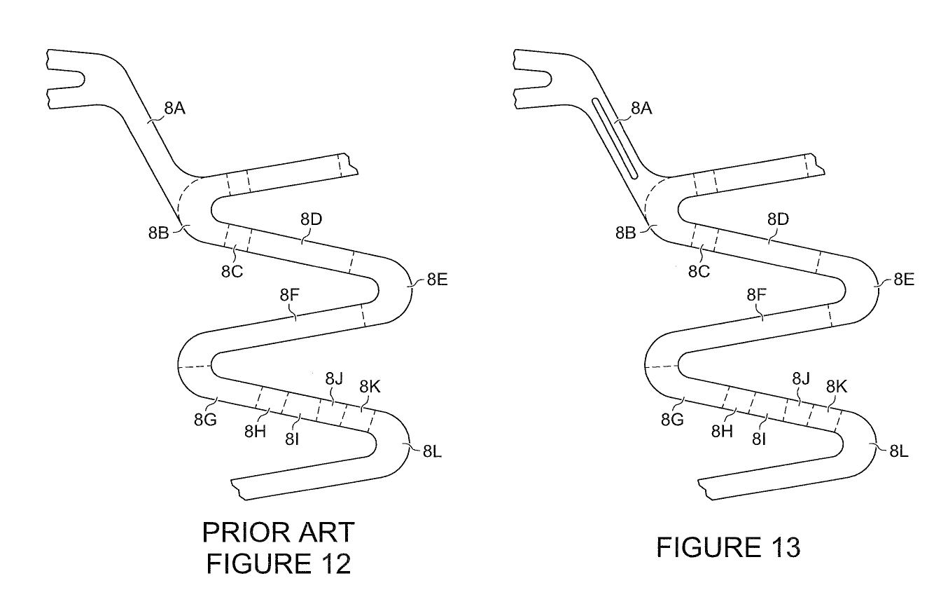

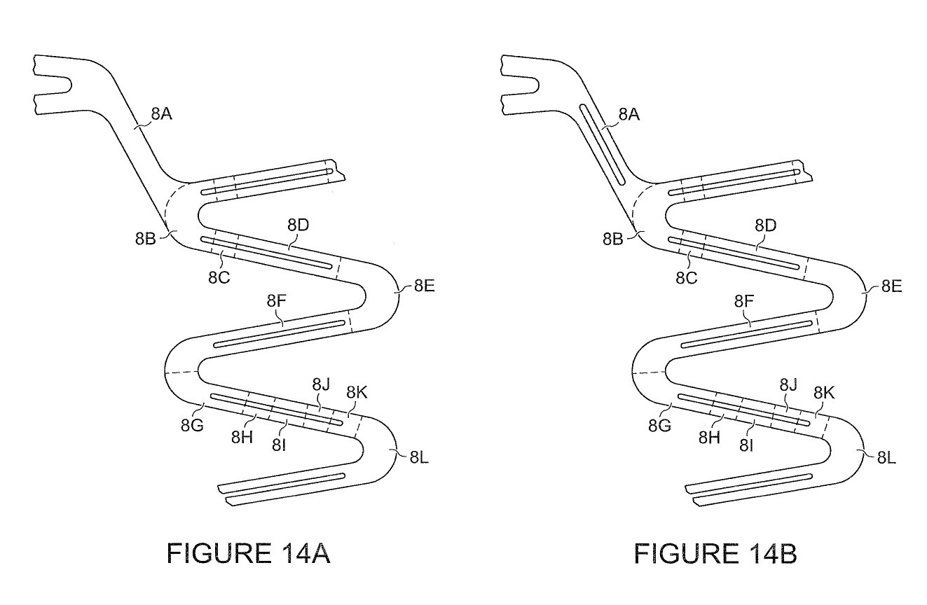

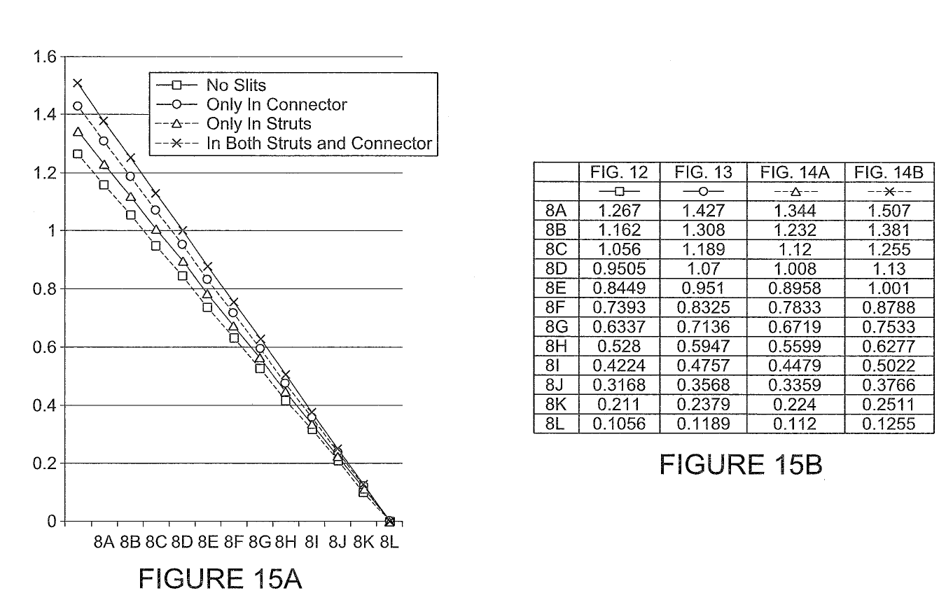

Referring now to FIGS. 12, 13, 14A and 14B there are shown how the stress levels on various stent segments change with slits in various adjoining stent members. In these figures, when an equal amount of radially directed expansive force is applied, the amount of resulting displacement is a direct result of the number of and position of various slits in the stent members. In each of these FIGs. the different segments of a stent are divided into segments 8A through 8L and each segment has a different displacement. The different displacements for each segment is tabulated in FIG. 15B and graphed in FIG. 15A. The units the displacements in FIG. 15B are measured in are in thousandths of a millimeter. The data shows that in those segments close to the connector, the presence or absence of a slit induces significant differences in displacement. Those areas undergoing the greatest displacement encounter the greatest stress levels.

FIG. 12 is a prior art stent with with no slits. In this design, the stress of expansion limits the expansion displacement of the stent members. In FIG. 13, a slit is positioned in a connector connecting two annular elements. The presence of the slit in the connector reduces the stress on the member when expanded allowing greater displacement. This reduction in stress reduces the amount of pressure needed to expand this member or conversely increases the extent to which it can expand without failing or shearing. The data in FIGS. 15A and 15B show that the presence of slits in the members of the annular element (as shown in FIG. 14A) reduces stress more than the presence of slits in a connector (as shown in FIG. 13). The data in FIGS. 15A and 15B show that the presence of slits in both the annular elements and the connector (as shown in FIG. 14B) results in a highly flexible structure with significant displacement potential.

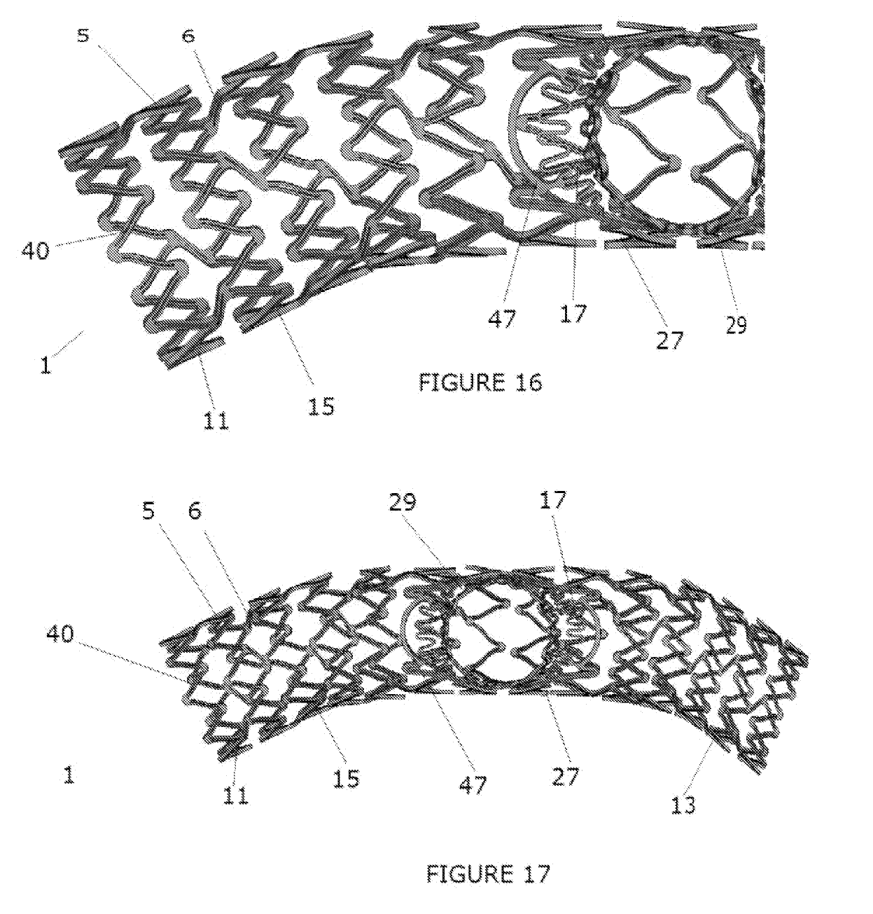

Geometry inherent in bifurcated stents can make use of these differing degrees of flexibility. Referring now to FIGS. 16, 17 and 18 there is shown a stent in a bent configuration which has slits in a number of its various regions. In FIG. 16 the bent configuration is characterized by a pitching bend at the proximal end of the stent. In FIG. 17 the bent configuration is characterized by a bend is a dextrally or rightward directed pitching bend at the proximal side of the stent (1). In FIG. 17 the bent configuration is characterized by a bend is a rightward directed pitching bend at both the proximal and distal sides of the stent (1). In both of these FIGs. It can be seen that because the ostial frame (47) encompasses a greater area than any one strut (5) or connector (6), a given bending force will be interfered with by the ostial frame (47) and will prevent as pronounced in the medial region (27) as in annular elements (11) in the distal (13) or proximal regions (15). Embodiments of the invention make use of this fact to use slits (40) to counteract or enhance the difference in flexibility between those of the distal (13) and proximal (15) regions and those of the medial (27) and ostial regions (17).

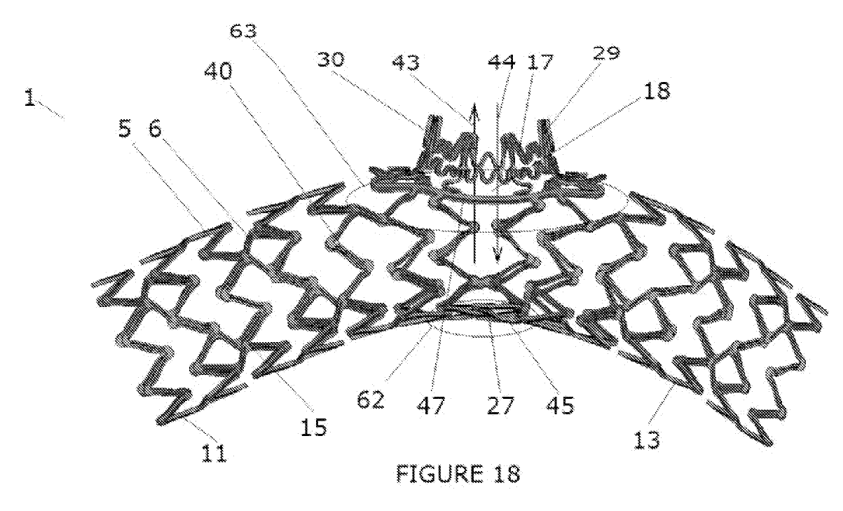

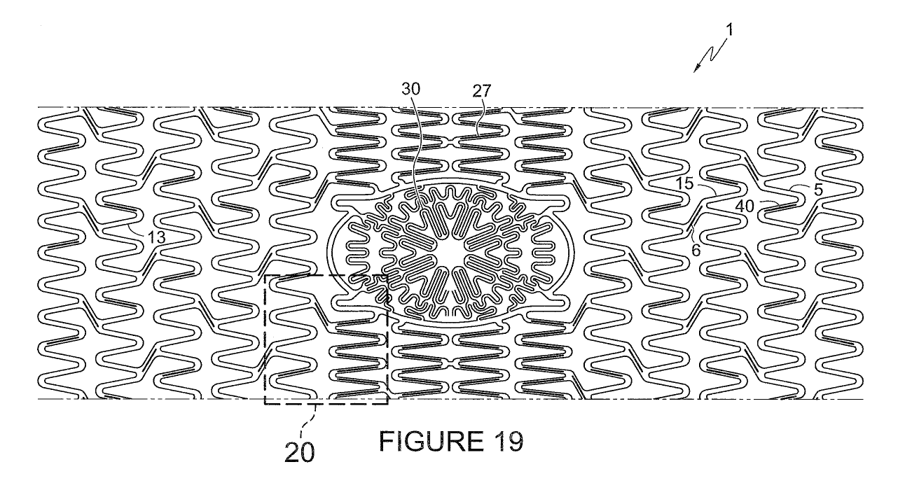

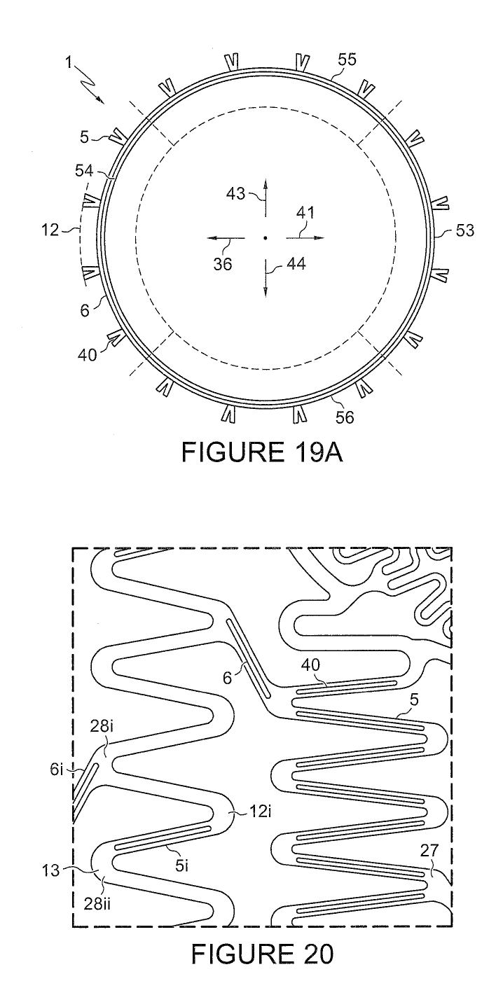

Referring now to FIG. 19 there is shown an embodiment in which the medial region is a high ratio portion of the stent because a higher proportion of the struts there have slits in them than in other regions of the stent (1). In at least one embodiment, the slits (40) are distributed within the struts (5) of the medial region (27), the connectors (6) of the distal (13) and proximal regions (15), and in select struts (5) of the distal (13) and proximal region (15). In the medial region (27), the slit (40) dispositions correspond to the stress levels illustrated in FIG. 15 and give the medial region (27) sufficient flexibility to at least partially counteract the rigidity of the side branch assembly (30). Such a configuration is particularly suitable for ventrally (44 of FIG. 18) directed yawing bends where the side branch assembly (30) is positioned at the apex of the bend (such as in the bend of FIG. 18) and the medial region (27) and in particular the portion of the medial region on the opposite side of the circumferential layer relative to the longitudinal axis of the stent (1) as the side branch assembly (45) will undergo significant “pinching”. For purposes of this application definition of the term “yawing” is displacement in the dorsal (43 of FIG. 18) or ventral (44 of FIG. 18) directions.

The definition of the term “pinching” is the movement of stent members closer together caused by the expansion of the stent. When one portion of a stent becomes pinched a portion (62) of the stent and the opposite stent side may also become a stretched portion (63). This can be seen in FIG. 18 where the portion of the proximal and distal regions adjoining the ostial region are stretched away from the ostial region but the portion on the opposite side of the circumferential layer as the side branch assembly is pinched closer the portion on the opposite side of the circumferential layer as the side branch assembly. The stretched portion (63) of the bend undergoes tensile stresses while the pinched portion (62) of the bend undergoes compressive stresses.

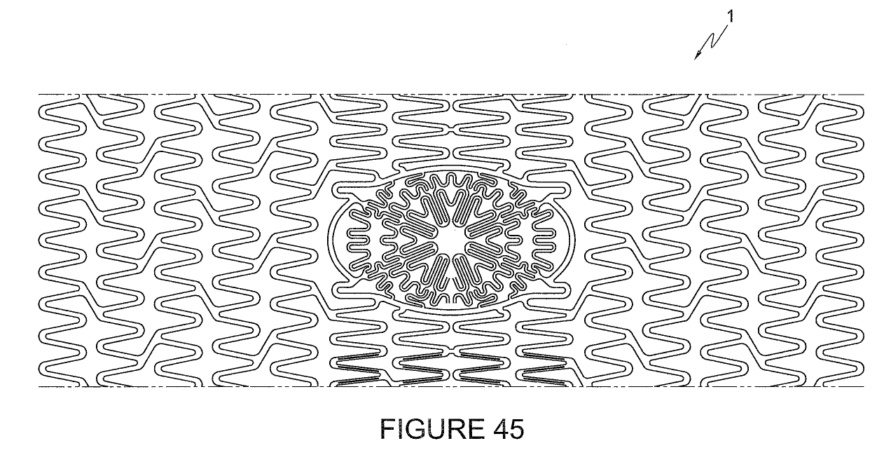

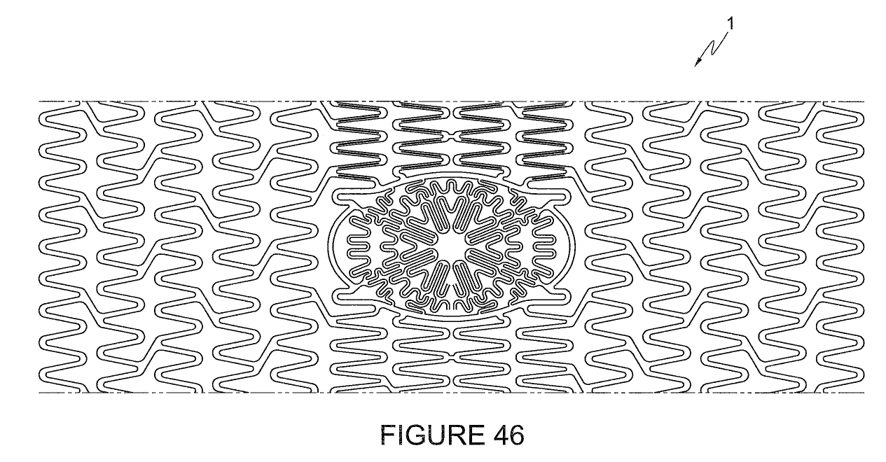

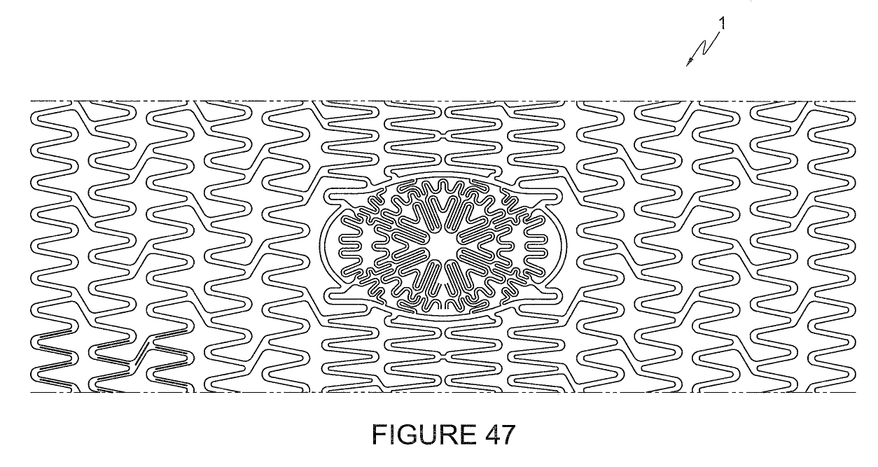

Referring now to FIG. 19A there is shown a circumferential cross section of a portion of the length of the stent viewed from the proximal side of the stent (1). The stent (1) has four quadrants (53, 54, 55, 56), the four quadrants being a left or sinistral quadrant (54), a right or dextral quadrant (53) diametrically opposite to the left quadrant, a dorsal quadrant (55) on the dorsal side of the cross section, and a ventral quadrant (56) diametrically opposite to the dorsal quadrant. In at least one embodiment the high ratio portion of the stent is within at least one quadrant. In the expanded state, the stent (1) bends with the pinched portion of the bend located at the quadrant having the high ratio portion, and the tensed portion of the bend located at the quadrant diametrically opposite the quadrant having the high ratio portion. In at least one embodiment some or all of one, or more than one quadrant has a high ration of slitted struts or slitted connectors. As illustrated in FIGS. 45, 46, and 47, the high ratio portion can be at any position on the stent. FIG. 45 illustrates an embodiment in which the high ratio portion is located at that part of the medial region which is on the opposite side of the side branch. FIG. 46 illustrates an embodiment in which the high ratio portion is on one side of the stent which will facilitate a bend in the stent. FIG. 47 illustrates an embodiment in which the high ratio portion is one quadrant in a portion of either the distal or proximal region.

Referring again to FIG. 19 it can be seen that the distal and proximal region connectors can also have slits (40) allowing them to flex more easily that the annular elements (11) of these regions. The slits (40) are positioned only on those struts (5) which do not adjoin any connector (6) and are circumferentially positioned between proximal and distal connectors. An example of this is shown in FIG. 20 where strut (5i) located between trough (28i) and trough (28ii). Trough (28ii) is engaged to strut (5i) and is not engaged to or is not otherwise adjoining any connector. Trough (28i) however is engaged to slitted connector (6i). Such a configuration allows for significant variability in the distance between the annular elements but prevents the annular elements themselves from becoming excessively expanded.

In at least one embodiment of a stent (1) with slits (40), an annular element (11) comprises alternating peaks (12) and troughs (28), and the connectors (6) are engaged to those peaks (12) and troughs (28). The connectors are engaged to every fourth peak (12) and to every fourth trough (28). The connected trough (28i) is between the third and fourth trough. A slit is positioned along the strut (5i) extending between the third peak (12i) and the second trough (28ii).

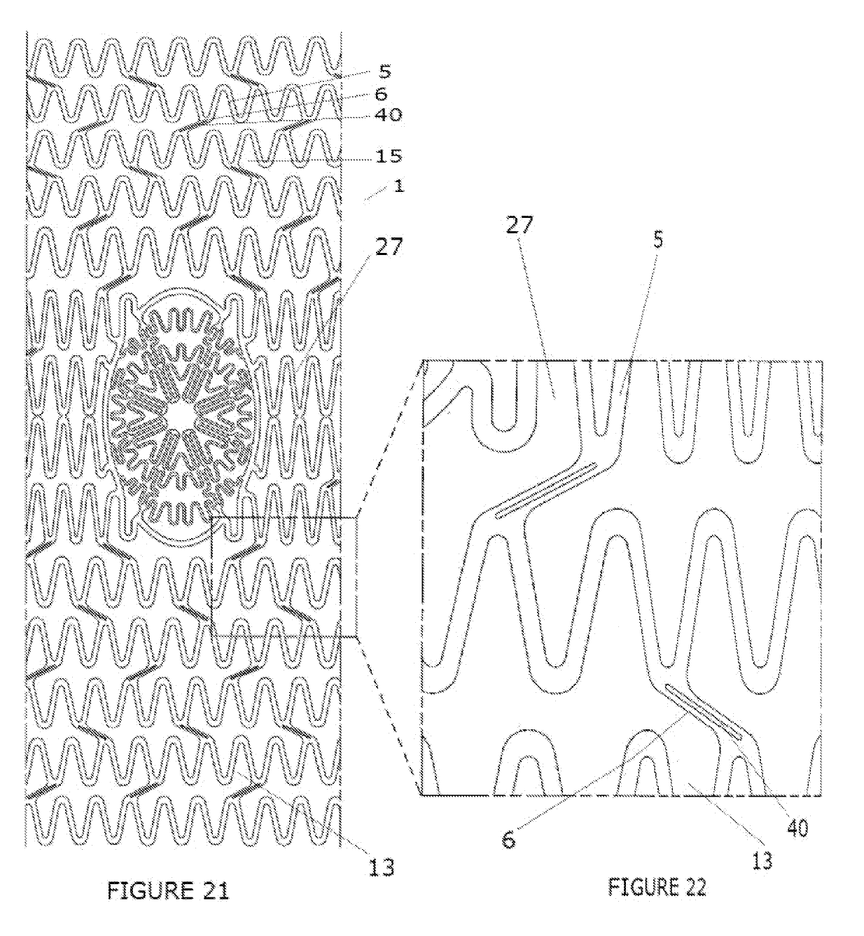

In one embodiment as shown in FIG. 21, slits (40) are only present in the connectors (6). This design results in a stent (1) capable of significant bending along its distal (13) and proximal regions (15) but that also has a generally rigid medial region (27). A close up view of this embodiment is seen in FIG. 22. This configuration is particularly suitable for stents undergoing yawing bending such as that illustrated in FIGS. 16 and 17.

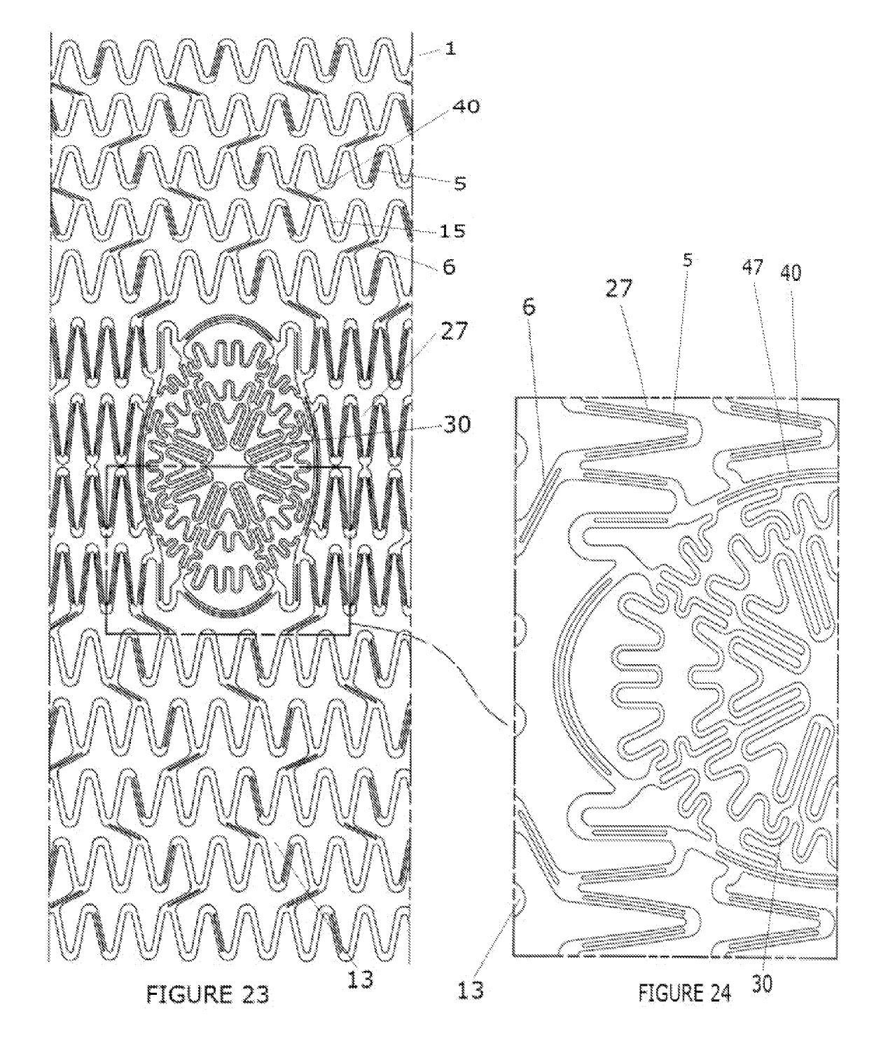

Slits (40) can also be positioned on the side branch assembly (30) itself Slits on the side branch assembly (30) allow the stent (1) as a whole to bend more and allow for an easier side branch expansion. FIG. 23 and close up FIG. 24 illustrate slits positioned in the ostial frame (47) of the side branch assembly (30). This embodiment is particularly suited for a stent undergoing a bend in which the side of the stent diametrically opposite to the side branch assembly will be the apex of the bend. In one embodiment, the slit (40i) is positioned across from the ostial connector (23) which connects the ostial frame (47) to other members of the side branch assembly (30).

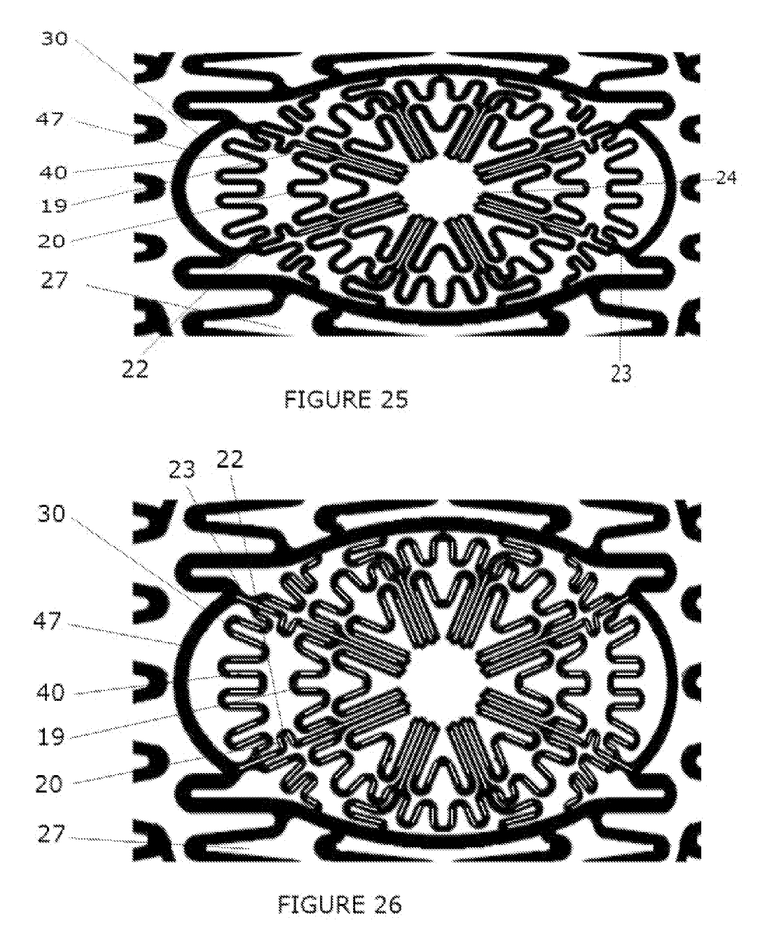

Referring now to FIGS. 25 and 26, the side branch assembly (30) comprises a plurality of generally linear spoke struts (19) generally extending from a position closer to the ostial frame (47) to a position closer to the oculus (21 of FIG. 3) of the extended side branch (29). The spoke struts (19) are interconnected by concentric rings (20). In the unexpanded state, the rings (20) are in an undulated configuration which at least partially straightens out in the expanded state. The spoke struts (19) provide radial scaffolding to the expanded side branch, and the concentric undulating rings (20) provide metal support and define at least some of the walls between the spoke struts (19). In one embodiment as shown in FIG. 25, the spoke struts (19) have slits (40) in them. This allows the spoke struts (19) to more easily bend and deploy while at the same time the concentric undulating rings (20) provide a strong side branch wall. The ostial connector (23) which connects the spoke strut (19) to the ostial frame (47) can be comprised from at least a portion of one or more spoke struts (19) and/or one or more of the concentric undulating rings (20).

In one embodiment as shown in FIG. 26, the spoke struts (19) and the concentric undulating rings (20) both have slits in them resulting in a highly flexible and easy to deploy side branch assembly (30). In at least one embodiment the spoke struts (19) comprise one or more turning portions (22) at those positions where the spoke struts (19) undergoes the most extreme amounts of bending stress. Detailed descriptions of turning portions (22) can be found in incorporated application Ser. No. 11/765679. In at least one embodiment the one or more turning portions (22) comprise two generally straight portions orthogonal to the majority of the spoke strut and are linked together by a curved portion. One or both of the straight orthogonal portions have one or more slits (40) in them. In at least one embodiment, the curved portion also has a slit in it and in at least one embodiment the slit extends through the entire turning portion (22). The slits (40) combined with the turning portions (22) result in a stent member with great flexibility.

In at least one embodiment the side branch is designed to extend at an angle which is non-perpendicular relative to the longitudinal axis of the stent resulting in one ostial connector (23) being bent at a most acute angle relative to all the other ostial connectors (23), one being bent at a most obtuse angle relative to all the other ostial connectors (23), and the remaining connectors (if any) being bent at intermediate angles. The bending stress is greatest on the most acute ostial connector (23), least on the most obtuse ostial connectors (23), and progressively increases with proximity to the most acute ostial connector (23). This non-perpendicular bending stress can be relieved by the positioning of one or more slits (40) along more acutely bent ostial connectors (23). In at least one embodiment, the slits progressively increase in length (along the length of the connector) and/or width relative to the connector's proximity to the most acute ostial connector (23).

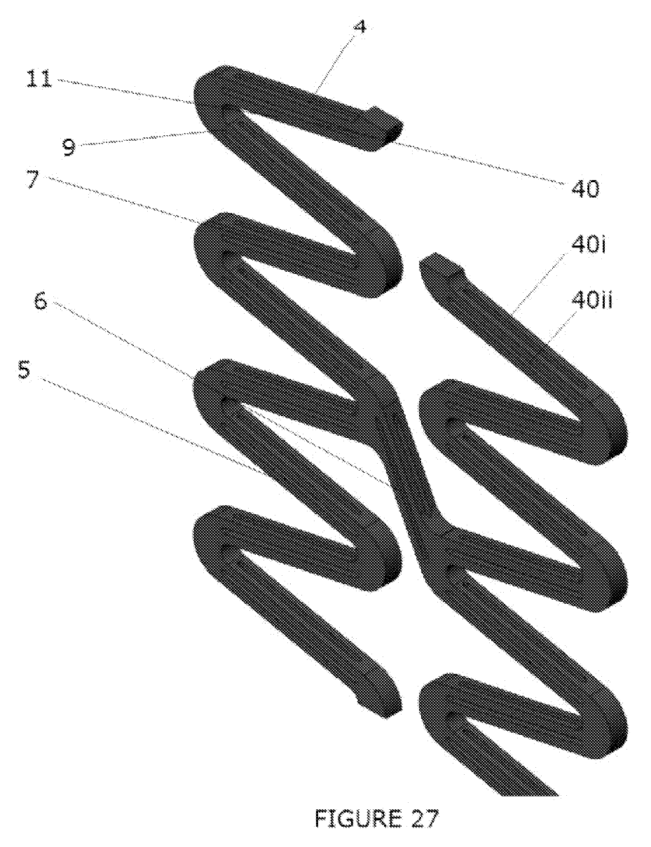

Referring now to FIG. 27 there is shown an embodiment of two annular elements (11) in which one or more struts (5) and/or one or more connectors (6) comprise one or more slits (40) that opens out to a position on a stent member that is neither the outer (9) nor inner surface (4) of the stent. For example slit (40i) extends through the width of the strut (5) it is within and does not open onto the outer (9) or inner surface (4) of the stent. FIG. 27 also shows that a slit can open up onto a stent member at any one, two, three or four sides of that member. For example slit (40ii) extends through the thickness of a strut and intersects with slit (40i) resulting in a strut having four slit openings.

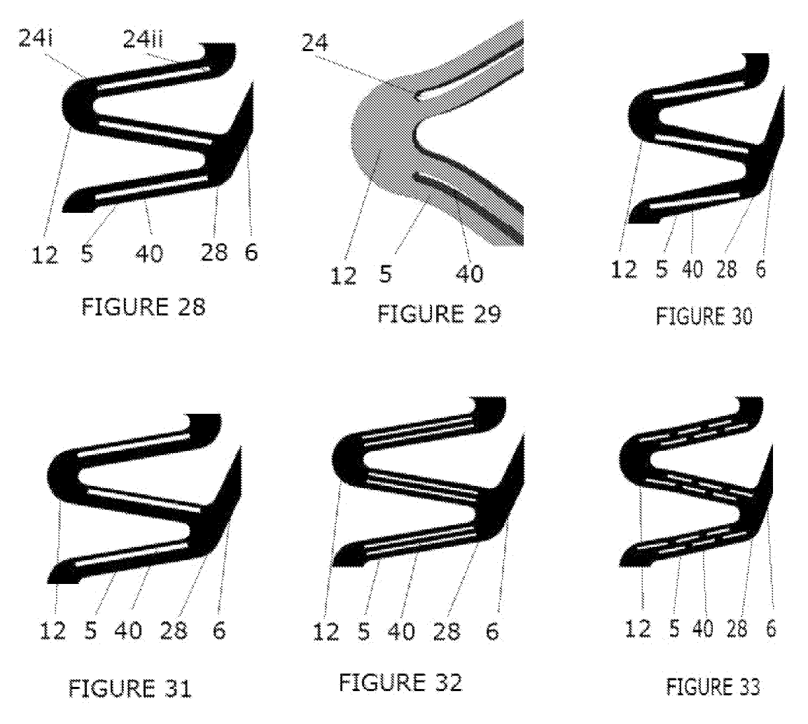

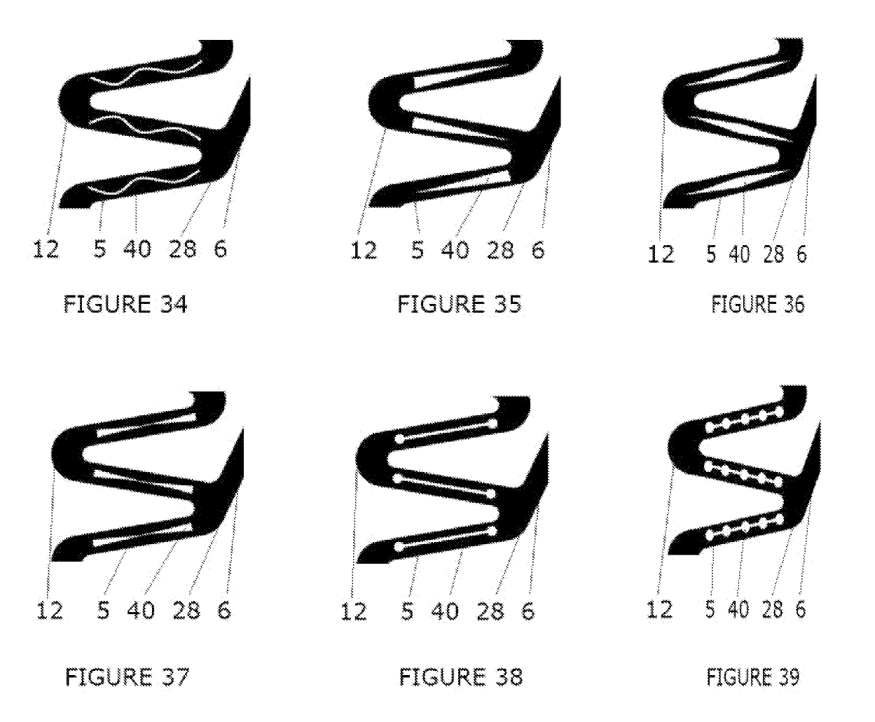

Referring now to FIGS. 28-39 there shown a number of different ways the slits (40) can be constructed. All of these embodiments contemplate slits (40) that extend completely or only partially through a given stent member and can be used in combination with each other on and along a number of stent members. FIG. 28 illustrates a slit (40) generally rectangular in shape extending between two ends (24i, 24ii) along a majority of the length of the strut (5). FIG. 29 illustrates a slit (40) in which the slit (40) has one or more diamond shaped ends (24). FIG. 30 illustrates a slit extending along an axis which is incongruent to the linear axis of the stent member it is within. FIG. 31 illustrates a slit offset away from the central axis of a member. Such positioning can be done, both to avoid the neutral axis of the member or to align the slit along the neutral axis of the member. FIG. 32 illustrates two or more slits positioned along the same stent member. These slits may be parallel or not. FIG. 33 illustrates more than one slit positioned in a linear series and shows that more than one series can be present (parallel or not) along the same stent member. The linear series can be aligned with each other or offset relative to the length of the member. FIG. 34 shows that the slits need not be linear and can extend along the member along an undulating or waved configuration. FIG. 35 shows a slit having a triangular configuration. FIG. 36 illustrates a trapezoid shaped slit, wider at the center of the slit and gradually narrowing at its ends. FIG. 37 shows an hourglass shaped slit narrower at the center of the slit and gradually widening at its ends. FIG. 38 shows a dumbbell shaped slit having sudden bulges at its ends. FIG. 39 shows a serial dumbbell shape having a number of sudden bulges along its length. The bulges can be consistently spaced or be positioned at irregular intervals.

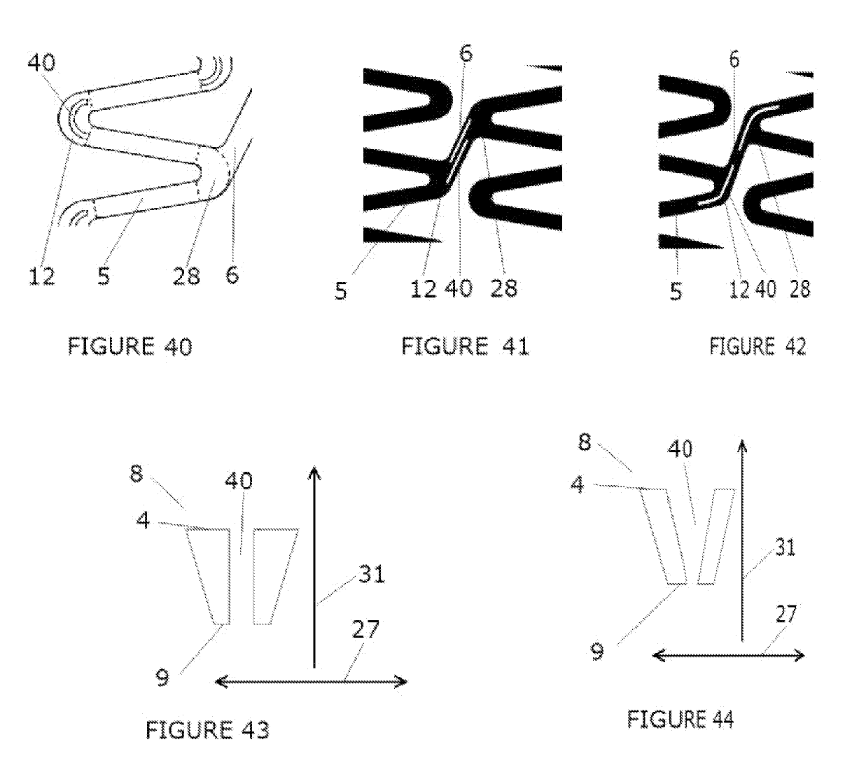

Referring now to FIGS. 40, 41, and 42 there are shown slits positioned along the connectors or peaks/troughs (12, 28) of an annular element. In FIG. 40, the slit curves along a peak/trough (12, 28). For purposes of this application, as shown by the phantom lines in FIG. 40, the border between a strut (5) and a peak/trough (12, 28) is the appropriately most distal or most proximal line that can be drawn along the width of a stent member in which the line forms a right angle with the sides of the stent member. Similarly phantom lines show that the border between a connector (6) and a peak/trough (12, 28) is that portion of the peak/trough-connector area that would not be encompassed by the superimposing of an adjacent peak portion over the peak/trough-connector area.

The curved slit (40) within the peak/trough (12, 28) can be aligned with the general path of the peak/trough (12, 28), or can be off center and be closer to one side or another of the peak/trough (12, 28). FIG. 41 shows a connector (6) extending from a peak (12) to a trough (28). The connector (6) has a pair of slits (40) extending thorough it. One slit (40) extends beyond the connector and into the peak (12) while the other slit (40) extends in the opposite direction and passes beyond the connector and into the trough. FIG. 42 shows a connector (6) having two serial slits (40) each of which extend beyond the connector (6) and into a strut (5) adjoining the peak or trough (12, 28).

The presence of slits in various stent members allows the stent when crimped about a catheter to be more easily deployed. The increased flexibility provided by the slits allows the stent to be compressed into a smaller cross sectional profile. This smaller size allows the stent to better fit into various body lumens than would otherwise be possible. In one embodiment, the slits are provided at the stent ends allowing the ends to be more flexible than other portions of the stent. These flexible ends reduce the effects of impact traumas against vessel walls the stent travels through. In addition, because the slits allow the stent as a whole to bend more, it is capable of traversing more obliquely shaped body vessels than would otherwise be possible.

The presence of slits on stent members also provides the stent with better body vessel wall apposition. Different portions of the stent can be programmed to have different magnitudes of allowed radial expansion. These differences can be made to specifically conform to the topography of the body vessel to be stented. If for example a body vessel has irregular topography due to shifts in plaque, calcium deposits, and or other causes, the stent can utilize slits to properly modulate the degree of expansion relative to the topography of the body vessel. In addition particular stent members can more easily bend or twist in response to the topography of the body vessel.

Referring now to FIGS. 43 and 44, there are shown cross sections of stent members (8) of varying width (27). For purposes of this application thickness refers to distance in a radial (31) direction. This is distinct from width (27) which refers to in a direction along the circumferential layer including along a circumferential and/or longitudinal directions. In at least one embodiment as shown in FIGS. 43 and 44, the stent members (8) have a tapering width (27) and are wider along the outer surface of the stent (4) than on the inner surface (9) of the member (8). These tapering widths allow for additional flexibility in the member (8). In at least one embodiment the stent member (8) is wider on its outer surface (4) than on its inner surface (9). As shown in FIG. 43, the width of a slit (40) can be independent of the width of a stent member (8). As shown in FIG. 44, the slit (40) can also correspond with the width of the member (8) resulting in a highly flexible stent member (8).

In some embodiments the stent, its delivery system, or other portion of an assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

The therapeutic agent can be at least one or various types of therapeutic agents including but not limited to: at least one restenosis inhibiting agent that comprises drug, polymer and bio-engineered materials or any combination thereof In addition, the coating can be a therapeutic agent such as at least one drug, or at least one other pharmaceutical product such as non-genetic agents, genetic agents, cellular material, etc. Some examples of suitable non-genetic therapeutic agents include but are not limited to: at least one anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof Where the therapeutic agent includes a polymer agent, the polymer agent may be a polystyrene-polyisobutylene-polystyrene triblock copolymer (SIBS), polyethylene oxide, silicone rubber and/or any other suitable substrate. It will be appreciated that other types of coating substances, well known to those skilled in the art, can be applied to the stent (1) as well.

This completes the description of the preferred and alternate embodiments of the invention. The above disclosure is intended to be illustrative and not exhaustive. This description will suggest many variations and alternatives to one of ordinary skill in this art. The various elements shown in the individual figures and described above may be combined, substituted, or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claims below.

1. A bifurcated stent having an unexpanded state and an expanded state, the stent constructed of a plurality of interconnected stent members, the stent comprising:
a generally tubular first body defining a first lumen, the first body having a side opening, two outer regions, one being a distal region distal to the side opening, and one being a proximal region proximal to the side opening, and a medial region between the outer regions which defines the side opening,
a second body comprising at least one projecting member, the second body in the unexpanded state positioned at least partially over the side opening, and in the expanded state bending at least in part away from the opening to define a second lumen in fluid communication with the first lumen,
at least one stent member has a slit characterized by an absence of material extending along a length of the member, the slit has a first side and a second side and is defined by a first sidewall on the first side and a second sidewall on the second side, when the sent is expanded, each of the sidewalls have a top, in the unexpanded state the two sidewall tops are substantially co-planar, in the expanded state they are non-co-planar.

2. The stent of claim 1 wherein the stent members the first body is constructed out of comprise a plurality of strut columns interconnected by connector members, each strut column comprises a plurality of strut members, each strut member of a strut column are connected at its proximal end by a peak to a first adjacent strut member and at is distal end by a trough to a second adjacent strut member, at least one connector member joins at least one peak of one strut column to one trough of an adjacent strut column.

3. The stent of claim 2 in which the at least one slit is located in a connector member that extends between a strut column of the medial region and a strut column of an outer region.

4. The stent of claim 1 wherein there are at least two slitted stent members and the at least two sidewalls of the at least two slitted stent members have a geometric displacement relative to each other in the unexpanded state which becomes deformed in the expanded state, the deformation of at least one of the geometric displacements of a slit sidewall being a compressing together of the sidewalls, and the at least one deformation of at least one of the geometric displacements of a slit sidewall being a being a staggering of the tops of the sidewalls.

5. The stent of claim 1 in which the slit sidewalls become at least partially twisted around each other.

6. The stent of claim 1 in which every connector member in one region has a slit.

7. The stent of claim 6 in which every connector member in at least one outer region is slitted and one strut member in each strut column of that same region is slitted, the slitted strut member is located between a peak and a trough neither of which is are engaged to a connector member.

8. The stent of claim 1 in which a plurality of strut members are slitted, and the strut members of a high ratio portion of the first body has a higher ratio of slitted strut members to non-slitted strut members than all other portions of the first body.

9. The stent of claim 8 which in the expanded state assumes a bent configuration, the stent portion having the highest ratio of slitted to non-slitted strut members becomes the pinched portion of a bend in the stent and that portion of the stent on the opposite side of a circumferential cross section of the that portion of the stent having the highest density of slitted strut members forms the tensed portion of a bend in the stent.

10. The stent of claim 9 in which the portion of the medial region on the opposite side of a circumferential cross section of the stent from the side opening is the high ratio portion.

11. The stent of claim 8 in which a circumferential cross section of a portion of a length of the stent has four quadrants, the four quadrants being a left quadrant, a right quadrant diametrically opposite to the left quadrant, a dorsal quadrant between the right and left quadrants on the dorsal side of the cross section, and a ventral quadrant diametrically opposite to the dorsal quadrant, the high ratio portion being within at least one quadrant, in the expanded state the stent bends with the pinched portion of the bend located at the quadrant having the high ratio portion, and the tensed portion of the bend located at the quadrant diametrically opposite the quadrant having the high ratio portion.

12. The stent of claim 1 in which at least one slit has one configuration selected from the list consisting of: rectangular shaped, rectangular shaped with diamond ends, serial rectangles, wavy, triangular, trapezoidal, hourglass shaped, dumbbell shaped, and serial dumbbell shaped.

13. The stent of claim 3 in which at least one slit extends past the end of a connector member and at least partially into at least one peak.

14. The stent of claim 1 in which there are no slits in any portion of the second body.

15. A bifurcated stent having an unexpanded state and an expanded state, the stent comprising:
a generally tubular first body defining a first lumen, the first body constructed out of a plurality of serially positioned strut columns interconnected by connector members, the strut columns are made up of strut members connected to adjacent strut members by peaks, the connector members joining at least one peak of one strut column to one peak of an adjacent strut column, the first body having a side opening, a distal region distal to the side opening, a proximal region proximal to the side opening, and a medial region between the distal and proximal regions, the side opening is defined by a rounded ostial frame which is engaged to at least one of the strut columns of the medial region,
a second body comprises a plurality of generally linear spoke struts engaged at a first end to the ostial frame and whose second end is positioned away from the ostial frame, spanning between the spoke struts are rings, in the unexpanded state the rings are in an undulated configuration and are positioned in concentric circles of differing distance from the ostial frame,
in the expanded state, the rings are at least partially straightened out, the spoke struts bend up away from the side opening, and the spoke struts and rings define a wall of a second lumen in fluid communication with the first lumen,
at least one spoke strut has a slit, the slit is characterized as an absence of material extending along a portion of a length of the spoke strut and opening onto at least one surface of the spoke strut, the slit at least partially defined by two sidewalls, each of the sidewalls extend from an outer surface at least partially down into the spoke strut,
in the unexpanded state the at least one slitted spoke strut has a first shape and in the expanded state has a second shape, the first shape being more linear than the second shape,
in the unexpanded state the two sidewall outer surfaces are substantially co-planar, in the expanded state they are non-co-planar.

16. The stent of claim 15 in which at least one portion of the ostial frame located non-inclusively between the engagement points with two spoke struts also has a slit extending through it.

17. The stent of claim 15 in which in the spoke strut has a turning portion positioned along the spoke strut length between the ring closest to the ostial frame and a next serially adjacent ring, the turning portion comprising two generally straight portions orthogonal to and engaged to the generally linear portion of the spoke strut and a curved portion linking the generally straight orthogonal portions, each of the generally straight orthogonal portions have a slit extending through them, another slit extends from a position on the spoke strut closer to the first end to one of the generally straight orthogonal portions and another slit extends from a position closer to the second end of the spoke strut to the other generally straight orthogonal portion.

18. A bifurcated stent having an unexpanded state and an expanded state, the stent comprising:
a generally tubular first body, the first tubular body defining a first lumen, the first body being constructed out of a plurality of strut columns interconnected by connector members, each strut column comprises a plurality of strut members, adjacent strut members being connected at a proximal end of the strut column at peaks and at a distal end of the strut column at troughs, the connector members joining at least one peak of one strut column to one trough of an adjacent strut column,
the first body comprising a first region, a second region and a medial region therebetween, the medial region including the second body,
the second body comprising a plurality of projecting members, in the unexpanded state the projecting members defining at least a portion of the first tubular body, in the expanded state the projecting members form the second body, the second body defining a side opening in the first tubular body and further defining a second lumen in fluid communication with the first lumen,
at least one connector member that extends between a strut column of the medial region and a strut column of at least one of the first region and the second region defining a slit, the slit is characterized as an absence of material extending along a length of the at least one connector member and opening onto both an inner and outer surfaces of the at least one connector member, along the length of the at least one connector member the slit being defined by two sidewalls,
in the unexpanded state the at least one connector member has a first shape and in the expanded state has a second shape, the first shape being different than the second shape,
in the unexpanded state the two sidewalls are substantially co-planar, in the expanded state they are non-co-planar.

19. The stent of claim 18 in which each connector member when in the expanded state has a neutral axis extending through at least a portion of the connector member, the neutral axis defining the locations on the connector member where the tensile and compressive forces imposed by the assumption of the expanded state cancel each other out, and the slit is positioned to be exactly centered along the neutral axis.

20. The stent of claim 18 in which the slit has a central axis extending through its middle along its length and each connector member when in the expanded state has a neutral axis extending through at least a portion of the connector member, the neutral axis defining the locations on the connector member where the tensile and compressive forces imposed by the assumption of the expanded state cancel each other out, the central axis is offset from the neutral axis by a proportion relative to the distance between the closest side of the connector member and the neutral axis.
